(19) United States
(12) Patent Application Publication
(10) Pub. No.: US 2010/0121285 A1
(43) Pub. Date: May 13, 2010

(54) MEANS FOR THE APPLICATION OF ACTIVE SUBSTANCES IN THE EAR CANAL OF A PATIENT

(76) Inventors: Oskar E. Illi, Belp (CH); Clarence P. Feldmann, Opfikon (CH)

Correspondence Address: PAULEY PETERSEN & ERICKSON, 2800 WEST HIGGINS ROAD, SUITE 365, HOFFMAN ESTATES, IL, 60169, US

(21) Appl. No.: 12/451,404

(22) PCT Filed: May 7, 2009
(86) PCT No.: PCT/CH2008/000209

§ 371 Date: Nov. 9, 2009

(30) Foreign Application Priority Data
May 7, 2007 (CH) 740/207

(51) Int. Cl. A61F 11/00; A61M 31/00

(52) U.S. Cl. 604/286; 604/285

(57) ABSTRACT

For applying medications in the form of active substances bound in a liquid or paste-like matrix in the ear canal, a means for absorbing and locally delivering a medication. The means includes a sack-like receptacle part in which a preferably textile tampon able to absorb the medication can be accommodated, and an elastic rubber retaining washer serving to fix the receptacle part in the outer ear. The receptacle part is firmly connected to a pulling device, such as a string, via the retaining washer. The retaining washer fixes the receptacle part in the outer ear canal and is thus supported on one side behind the anthelix and on the other side behind the tragus. In one embodiment, the retaining washer is made from a flat material stamped from foam rubber. The applicator can be sold with a charge of medication.

### BACKGROUND OF THE INVENTION

1. Field of the Invention

This invention relates to applying active ingredients bound in a liquid or paste-like matrix in an auditory canal of a patient.

2. Discussion of Related Art

In many ailments in the region of the outer auditory canal, of the tympanic membrane, or of the middle ear, with perforations of the tympanic membrane, medicaments having active ingredients, which are bound in a liquid or paste-like matrix, are applied in the outer auditory canal. While medicaments having a liquid basis are most often applied as so-called “ear drops” directly into the outer auditory canal, medicaments having a paste-like basis are today conventionally applied in the outer auditory canal in the form of a tampon. This application does not occur by patients themselves, but rather is carried out by a treating physician. In so doing, the physician conventionally prepares a gauze strip that is saturated with the desired medicament that is subsequently wound into a tampon and applied in the outer auditory canal. Conventionally, patients require a plurality of tampons prior to complete healing. This requires a plurality of visits to the physician.

The application of liquid medicaments in the auditory canal is primarily effected today by self-administration. However, the self-administration is not without problems. For this purpose, medicine drop bottles are available on the market by which dispensation is possible by drops. Medicine drop bottles are, however, highly unsuitable for a self-administration of the medicament. For such self-administration, the patient must hold the bottle in the proper position distanced over the opening of the auditory canal, subsequent to which the patient must administer the proper number of drops, based on feeling. This must take place without contact since otherwise an uncontrolled outflow would be effected. However, should the patient hold the medicine drop bottle in a distanced manner, it is still not ensured that the drops will be applied at the proper location. Furthermore, such an administration is possible practically in a lying-down position only.

Also, medicine bottles are available on the market of which are closed with a pipette. In the filled state, the pipette customarily contains too great of an amount of the liquid medicament. Thus, if the patient empties the pipette entirely, the patient receives an overdose. With outside help, the medicament can be dispensed by a pipette without any problem, yet this is hardly possible in the instance of self-administration. If the pipette merely comes into contact with the ear, the medicament then practically flows out entirely in an instant. However, if one desires to empty the medicament from the pipette by drops without the pipette coming into contact with the ear, one encounters the problem that it is practically impossible to aim correctly. The drops thus arrive somewhere in the auricula and the likelihood that a portion of the applied amount of the medicament never enters into the auditory canal is correspondingly large. The result is an underdose of the medicament. Furthermore, bottles are also available on the market that have a rubber bushing, wherein the medicament can be dispensed in drops by squeezing the bushing. Likewise with this solution, the problem remains the same.

If after administration of eardrops the patient does not remain lying on the same side, then the administered eardrops flow out again. This also occurs even if the patient goes from a horizontal position to a vertical position. Not only is this uncomfortable for the patient, but it also results in an underdose of the medicament.

In the end, it is also uncomfortable for the patient that the ear, which has been moistened by the ear drops, is not protected. Correspondingly, the patient notices with pronounced discomfort any form of draft. In many cases patients protect themselves by preparing cotton balls themselves that they then plug into the auditory canal. Some patients also saturate cotton balls with corresponding liquid medicaments and push them into the auditory canal themselves. Otologists, however, warn against such practices but still practices continue to occur just as frequently. It is not uncommon that the cotton ball is pushed too deeply into the internal auditory canal, after which it can no longer be pulled out without an instrument, while when they remain in place, they can lead to irritations of the tympanic membrane. With the removal by instruments such as, for example, pincers or substantially unsuitable instruments, damage to the external auditory canal or even to the tympanic membrane continue to arise.

### SUMMARY OF THE INVENTION

Thus, one problem addressed by this invention is to provide a suitable application of active ingredients bound in a liquid or paste-like matrix in the auditory canal of a patient while avoiding the previously-mentioned problems and while avoiding the risk of causing damage and erroneous dosing.

This problem is solved by a means having features described in this specification and in the claims. Additional advantageous embodiments proceed from the dependent claims, and their meaning and mode of action is described in the following description by reference to the attached drawings.

### BRIEF DESCRIPTION OF THE DRAWINGS

One embodiment is shown in the drawings and is described in this specification, wherein:

FIG. 1 is a perspective view of a first embodiment of means for the application or an applicator of active ingredients in the auditory canal;

FIG. 2 is a view of the same means on an opposite view of an external side, in the use position;

FIG. 3 shows a second embodiment of the means in a vertical, longitudinal section;

FIG. 4 shows a view of the means according to FIG. 3, from a side in the use position;

FIG. 5 shows an alternative design in the representation as shown in FIG. 4;

FIG. 6 shows a simplified representation of the means according to this invention applied in the external auditory canal in an anatomical section; and

FIG. 7 shows the use of the means with a view of the ear from the perspective of the user.

### DETAILED DESCRIPTION OF THE INVENTION

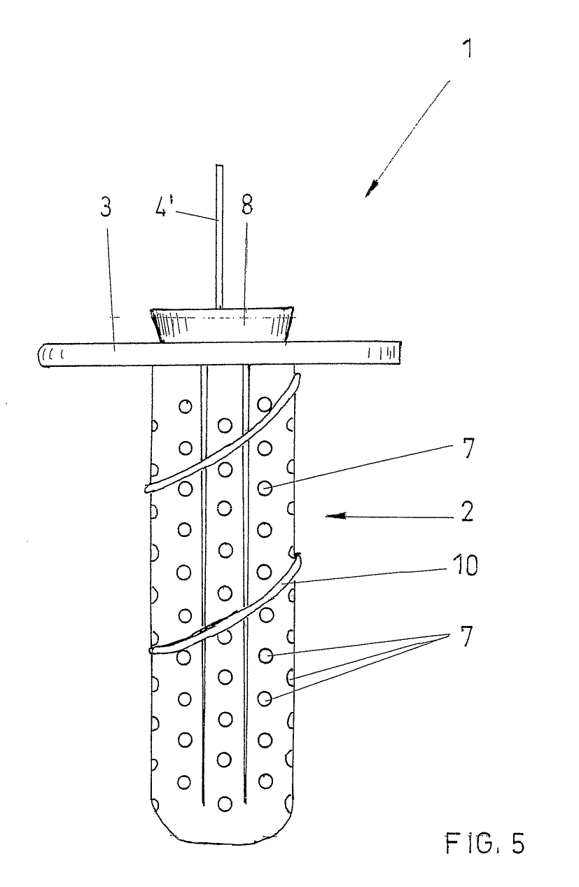

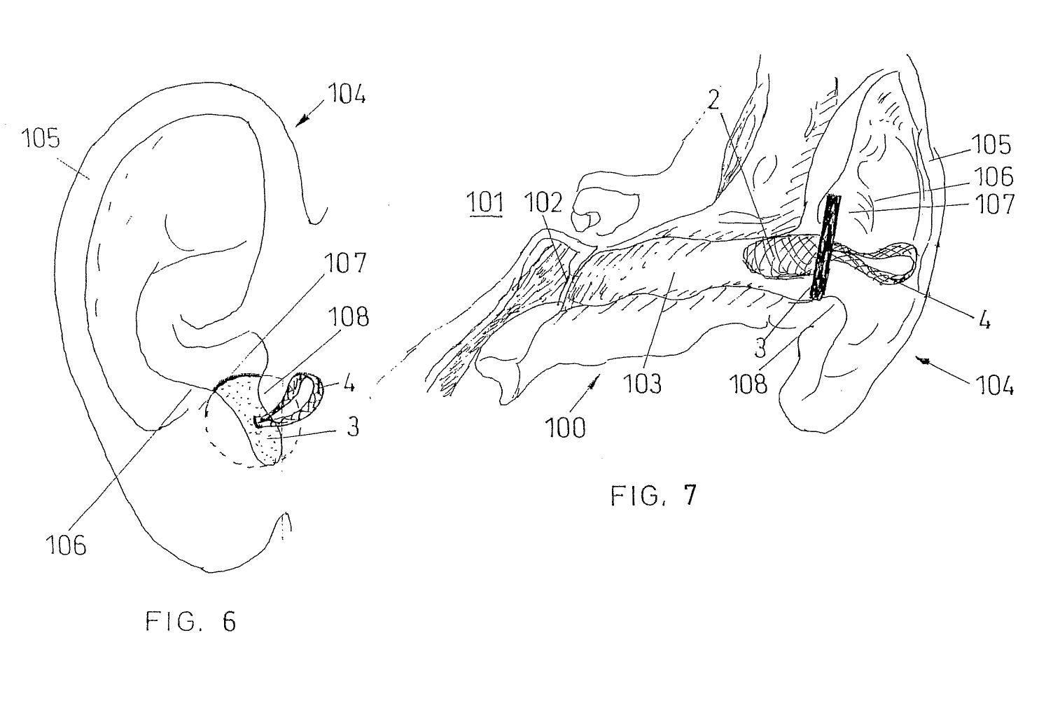

First, with reference to FIGS. 1-4, two different preferred embodiments of this invention are addressed, subsequent to which their use is explained using FIGS. 5 and 6.

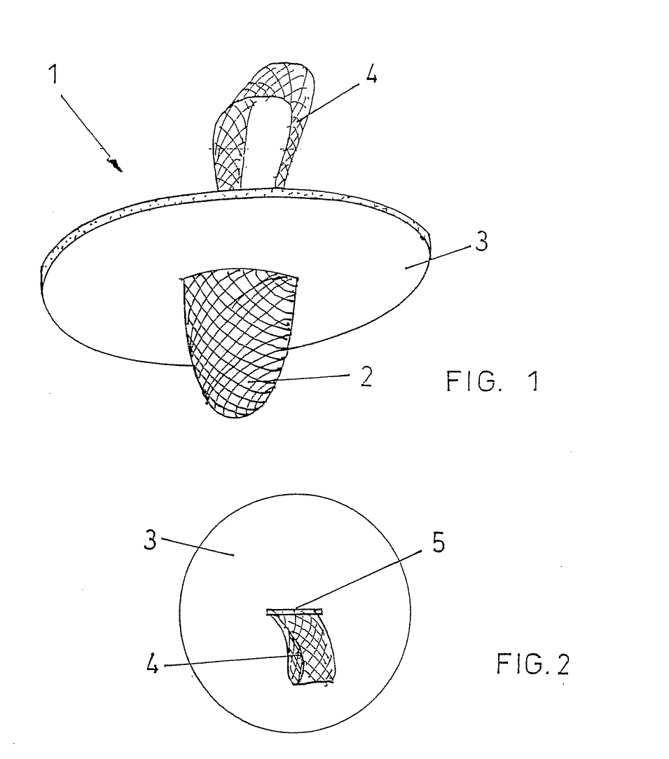

The means for the application of active ingredients bound in a liquid or paste-like matrix in the auditory canal of a patient, which embodiment is particularly simple, economical, and effective in clinical trials, is shown in one embodiment according to FIGS. 1 and 2. The entire means, which is also known as an applicator, is identified by reference number 1 and comprises a sack-like receiving portion 2, a retaining disc 3, and a pulling member 4 that passes through the retaining disc 3. The sack-like receiving portion 2 and the pulling member 4 are preferably integrally connected to each other. Although the pulling member 4 is shown in the embodiment here as a loop of gauze material, a simple cord could be present instead that must be firmly attached to the sack-like receiving portion 2.

The shaping of the sack-like receiving portion 2 corresponds to a dispensing tampon in construction. It contains an absorbent core composed of cotton, products made of viscose or a latex-free plastic, for example, that are held together by gauze that forms the receiving portion. This gauze can, for example, be composed of a closed tube section the end of which also forms the pulling member 4 at the same time. The manufacture is substantially dependent upon the available manufacturing means.

The formation of the retaining disc 3 is of primary importance for the functional capability of the entire applicator. The retaining disc 3 fulfils, in particular, the following functions.

First, the retaining disc 3 ensures that the tampon present in the receiving portion does not get lost in the external auditory canal. Second, it provides protection against the medicament flowing out from the external auditory canal. Third, it insulates from cold and convection. Fourth, it insulates from sound. Fifth, it secures the applicator in the external auditory canal by a lodging in the ear ossicles.

The diameter and the shape of the external auditory canal in cross-section differ greatly from person to person. As testing has shown, it is thus important for the retaining disc 3 to be satisfactorily adjustable. If this is indeed the case, the shaping of the retaining disc 3 is practically of secondary importance. In a particularly economical embodiment, the retaining disc 3 can be punched out of a sponge rubber sheeting. This material is of great comfort for the wearer, adjusts extraordinarily well to the shapes of the auricula, and has only a relatively minimal restoring force so that it is not by the restoring force alone that the retaining disc 3 is inclined to pull the receiving portion 2 with tampon from the auditory canal. It has been demonstrated in experiments that sponge rubber sheeting having a thickness of 1-3 millimetres is particularly suitable. Preferably, a sheeting thickness of approximately 1.5 mm is selected. Sponge rubber is understood as microcellular expanded rubber manufactured from latex or a latex-similar plastic. This material, which is offered in sheeting, can easily be punched into the desired shape. In so doing, both the external shape as well as a slit 5, which is suitable for feeding the loop 4 through, can be punched at the same time.

Because the tampon in the receiving portion is saturated with an oily or a pasty preparation, the pulling member 4 could likewise become soaked and cause a corresponding capillary suction effect. This presents no problems in and of itself but this is uncomfortable for the wearer. In order to prevent this, the pulling member 4 can be correspondingly coated. If manufactured out of gauze, the pulling member 4 can then be coated with wax or stearin, which would counteract this capillary effect. Additionally, because the pulling member 4, in particular if manufactured out of gauze, maintains a greater degree of rigidity, it can be grasped again in a more favorable manner.

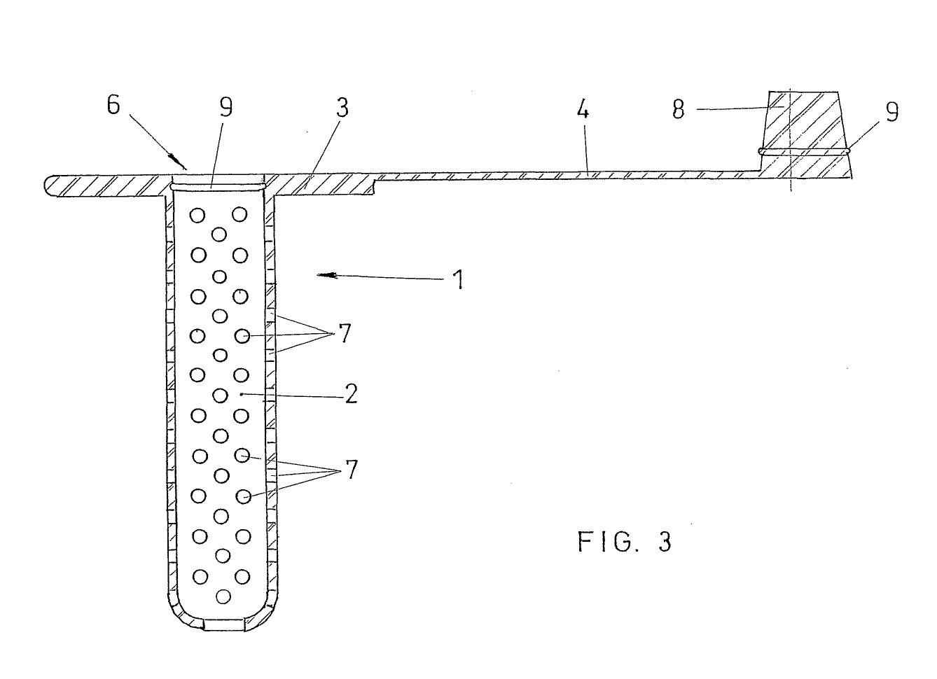

With regard to the initial investment outlay in the manufacturing process, the solutions shown in FIGS. 3 to 5 are those in which the entire means are manufactured from latex-free plastic. The means 1 comprises the same parts as the previously described variants.

The main component is the sack-like receiving portion 2, the open end of which is integrally molded with the retaining disc 3. The retaining disc 3 has an insertion opening 6. The insertion opening 6 forms the open end of the sack-like receiving portion 2 at the same time. Because the entirety of the means can be of plastic, it exhibits sufficient inherent rigidity in order to be subsequently filled with a tampon. The tampon can be configured out of a cotton roll or foam rods that fit in the receiving portion 2 with minimal interference. Only if the tampon is positioned relatively pressure-free in the receiving portion can it optimally fully absorb active ingredients dissolved in a fluid matrix. At the same time, an optimal adaptation to the individual, anatomical shape of the auditory canal is achieved due to the compressibility of the tampon.

However, experiments have shown that with a plastic configuration, the receiving portion need not necessarily be filled with a tampon. If the external side of the receiving portion comes into contact with the active ingredient bound in a liquid matrix, the receiving portion 2 becomes filled through the perforations 7 by capillary suction.

Ideally, the means can be provisioned or supplied with active ingredients by patients themselves. If one moves the pipette along the sack-like receiving portion 2 that is provided with perforations 7, the receiving portion 2 fills up and the means can be inserted into the auditory canal. The respective daily dose/daily doses can thus be applied in a visually controlled manner.

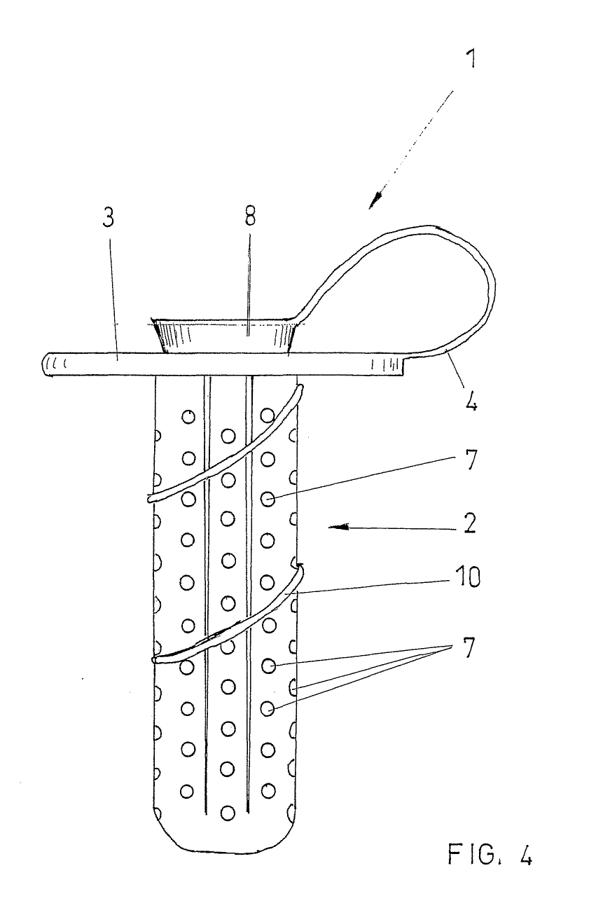

In the embodiments according to FIGS. 3 and 4, the pulling member 4 is directly molded on the retaining disc 3. Additionally, a finger is integrally formed on the pulling member that is configured as a cord, with which finger the insertion opening 6 can be closed. In order for the finger to remain in place, the taper of the finger 8 is alone not sufficient. Therefore, a circumferential, annular groove or bead is integrally molded on the finger, which groove or bead cooperates with a diametrically opposed shape on the internal side of the sack-shaped receiving portion 2 close to the insertion opening 6. A combined form-fitting and non-positive connection of the finger 8 in the receiving portion can thus be effected.

If desired, the external side of the receiving portion 2 can be stiffened by braces 10. The braces 10 can extend helically or longitudinally. It is practically possible to form the sack-shaped receiving portion 2 from a network of helical and longitudinal braces, wherein the remaining intermediate spaces form the perforations 7. This corresponds to the mesh-like structure of gauze. The gauze has intermediate spaces between the weft threads and the warp threads that form the required openings.

For injection-molding technical reasons, it is advantageous to configure the finger 8 separate from the receiving portion 2. This requires less extensive surface of the injection mold, which in turn reduces the investment costs.

In order to ensure that the patient does not undertake any undesired manipulations of the means, it is additionally possible to provide the finger 8 with a serrated bead 9 as a securing means, wherein a diametrically opposed annular groove is provided in the receiving portion. In this instance, the receiving portion 2 is closed by the finger 8 by the manufacturer subsequent to manufacturing and can no longer be opened. An additional securing means of this connection can also be achieved by ultrasonic sealing, heat sealing or adhesion. This guarantees that the user does not remove the cotton or foam plug, if present, in order to saturate it. The saturation of the plug can now only take place from the outside and thus a contamination of the plug is virtually impossible.

For purposes of handling, the finger 8 has a highly flexible, thin-walled tab 4′ as a pulling member. Nevertheless, the finger cannot be pulled out or at least not unintentionally. The tab and the finger can also be manufactured from a material that is different from the rest of the means. With the irreversible connection of the finger 8 to the receiving portion 2, these two parts are also connected to one another.

The use of the means according to this invention is represented in FIGS. 6 and 7. The entire external ear is referenced with reference number 100. The inner ear, which is not shown in detail, is referenced with reference number 101. A separation between the inner ear 101 and the external ear 100 is effected by the tympanic membrane 102. The tympanic membrane 102 borders the external auditory canal 103. The auricula 104 is bordered by the helix 105, the so-called edge of the auricula. Running approximately parallel to the helix is an arced ridge, the antihelix 106. Opposite the antihelix 106 is a cartilaginous protrusion in front of the external auditory canal opening. This cartilaginous protrusion is characterized as a tragus 108. The antihelix 106 and the tragus 108 are positioned on the edge of the concha 107 that represents a recess that leads into the external auditory canal. Although the shape and the cross-section of the external auditory canal 103 are relatively different, one can get along with only one length of the receiving portion. The additional adjustments to the ear occur through the flexibility of the retaining disc 3 and permit a relatively large adjustment, while also remaining in place behind the concha 107 and the tragus 108 and the antihelix 106. In the plurality of all possible cases, one will be able to use a means according to this invention. In theory, an enlarged concha 107 is present in only less than 1% of all patients, while at the same time the tragus 108 is not present or is only rudimentarily present. Also, because in all mandibular movements the cross-section of the external auditory canal is changed, the dispensation of the medicament is thus favored.

The applicators according to this invention can be delivered without medicaments directly to otologists and patients alike, both of whom respectively provide the tampon or the receiving chamber with the medicament. It is also entirely possible to put directly on the market the means according to this invention that have already been provided with medicaments.

1. A means for application of active ingredients bound in a liquid or a paste-like matrix in an auditory canal of a patient, wherein the means (1) comprise a sack-like receiving portion (2) having a perforated structure for receiving the bound active ingredients, and a retaining disc (3) holding the means (1) in the auditory canal that can grip from behind the concha (107) and/or the tragus (108), and a pulling member (4) that is connected directly or indirectly to the receiving portion (2) and that serves for the insertion as well as removal of the means (1) from the auditory canal, comprising the sack-like receiving portion (2) and the retaining disc (3) and the pulling member (4) manufactured from plastic, and a closeable insertion opening (6) in the retaining disc (3) forming access means to the receiving portion (2).

2. The means as specified in claim 1, wherein the pulling member (4) is a tab that is integrally formed on the retaining disc (3).

3. The means as specified in claim 1, wherein the pulling member (4) has a terminal finger (8).

4. The means as specified in claim 3, wherein the finger (8) is measured so that it can be inserted so that it is held in the insertion opening (6) of the sack-like receiving portion (2), wherein the pulling member (4) forms a loop that protrudes from the plane of the retaining disc (3).

5. The means as specified in claim 1, wherein the sack-like receiving portion (2) has braces (10) that extend helically on the external side.

6. The means as specified in claim 1, wherein the sack-like receiving portion (2) has a plurality of regularly arranged holes or perforations (7).

7. The means as specified in claim 1, wherein the sack-like receiving portion (2) is filled with an absorbent cotton or foam string or plug.

8. The means as specified in claim 1, wherein the finger (8) is manufactured from the receiving portion (2) and is held in a conditionally detachable manner by securing means (9) in the receiving portion (2).

9. The means as specified in claim 8, wherein the pulling member (4) is configured as a thin-walled, highly flexible tab (4′).

10. The means as specified in claim 8, wherein the finger (8) and the tab (4′) are manufactured out of plastic that is different from a remainder of the means (1) for the application of the active ingredient.
